(19) United States
(12) Patent Application Publication
(10) Pub. No.: US 2006/0008507 A1
(43) Pub. Date: Jan. 12, 2006

(54) System for generating a bioactive dosage form

(76) Inventor: Makarand P. Gore, Corvallis, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/885,054

(22) Filed: Jul. 6, 2004

(51) Int. Cl. A61K 47/00

(52) U.S. Cl. 424/439

(57) ABSTRACT

A system for generating a bioactive dosage form, including a first drop-on-demand fluid ejector fluidically coupled to a first reservoir that contains a first fluid having a first reactant. The first drop-on-demand fluid ejector is capable of ejecting a drop of the first fluid onto a pre-selected location of an ingestible substrate. In addition, the system also includes a second drop-on-demand fluid ejector fluidically coupled to a second reservoir that contains a second fluid having a co-reactant which reacts with the first reactant. Either the first fluid or second fluid contains a bioactive agent.

### BACKGROUND

Description of the Art

The precision dispensing of a bioactive material plays an important role in such diverse areas as pharmaceutical, agricultural, chemical, and food industries to enhance the effectiveness of a particular component at the lowest possible cost. For example, the oral administration of pharmaceuticals is one of the most widely utilized methods to provide effective therapy for a variety of illnesses. The release of orally administered medications may occur in the oral cavity such as for buccal or sublingual administration, or it may occur in the gastrointestinal tract after the oral dosage form is swallowed. There are, for example, capsules and tablets designed to release an active ingredient in the stomach, enteric-coated formulations that release the medication in the intestinal tract of the patient, and controlled release dosage capsules that release the drug in both the stomach and the intestines. In addition, many individuals suffer from chronic health problems that require the regular administration of medicaments. Diseases such as diabetes, allergies, epilepsy, heart problems, AIDS, and even cancers require the regular delivery of precise doses of medicaments if patients are to survive over long periods of time.

Many pharmaceutical doses in tablet, capsule, or liquid form are made in formulations of a predetermined quantity of pharmaceutical units in each dose. Unfortunately, conventional oral dosage forms suffer from a number of disadvantages. Typically, to effectively handle and dispense small doses a considerable amount of adjuvant material must be added in order that the final dosage form is of a manageable size. Thus, typical methods for manufacturing include the mixing of the pure drug with various other substances commonly referred to as excipients or diluents that are therapeutically inert and acceptable by regulatory bodies, such as the Federal Drug Administration (FDA). In addition, the profile and kinetic pattern governing the release rate of an active component is difficult to control. For example, in the utilization of microcapsules, many if not most micro-encapsulation techniques generate a broad distribution of microcapsule sizes. The broad distribution in microcapsule size makes it more difficult to accurately dispense an optimal drug dosage as well as producing greater variability in dissolution rates and thus decreases the control over the absorption rate of the drug in the body. Further, there is an increasing need to control the drug absorption process to sustain adequate and effective drug levels over a prolonged time period.

The availability of useful drug delivery systems that provide an optimal drug dosage to be delivered by means of a precision dosage form is very limited. The ability to control and extend the release of an active component from a dosage form without adversely modifying the structure or normal biological function of the active component in the body after administration and absorption is also extremely limited today. If these problems persist, many new and potentially life saving beneficial drugs will either be impractical or have limited effectiveness in the dosage forms currently available. As the demands for more efficient and lower cost drugs continues to grow, the demand to develop systems or drug carriers capable of delivering precise amounts of the active ingredient, while increasing the therapeutic efficacy will continue to increase as well.

### BRIEF DESCRIPTION OF THE DRAWINGS

FIG. 1 is a cross-sectional view of a fluid ejection device according to an embodiment of the present invention.

FIG. 2a is a graph illustrating a normalized drop-size distribution of a conventional fluid ejector.

FIG. 2b is a graph illustrating a normalized drop-size distribution of a fluid ejection device according to an embodiment of the present invention.

FIG. 3a is a plan view of a portion of a dosage form according to an embodiment of the present invention.

FIG. 3b is a cross-sectional view of a portion of a dosage form according to an alternate embodiment of the present invention.

### DESCRIPTION OF THE PREFERRED EMBODIMENTS

The present invention advantageously utilizes a fluid ejection system to eject a drop of a precise volume of a first fluid, that includes a reactant material, onto a pre-selected location of the surface of an ingestible substrate. The fluid ejection system also ejects a drop of a precise volume of a second fluid over the drop of the first fluid where the second fluid includes a co-reactant, which reacts with the reactant in the first fluid either on contact or after subjection to additional energy. In addition, either the first or second fluid also includes a bioactive substance. The present invention may utilize a wide variety of drop-on-demand types of fluid ejection devices. For example, thermally activated fluid ejection devices, piezoelectric, and acoustic activation as well as others may be utilized in the present invention. The present invention provides both for smaller drop volumes as well as greater control over repeatability of drop volume with its corresponding narrower distribution of drop volumes than typical fluid dispensing techniques.

For purposes of this description and the present invention, the term “bioactive” as used with fluid, composition, substance, or agent, includes pharmacologically active substances that produce a local or systemic therapeutic effect in animals. The term includes active substances that affect a biological function of vertebrates directly or as a result of a metabolic or chemical modification associated with the organism or its vicinal environment. For example, a bioactive fluid may include any pharmaceutical substance, such as a drug, which may be given to alter a physiological condition of a vertebrate, such as a disease. A bioactive fluid is meant to include any type of drug, medication, medicament, vitamin, nutritional supplement, or other compound that is designed to affect a biological function of a vertebrate. The term includes any substance intended for use in the diagnosis or therapeutic treatment or prevention of disease. The term animal includes humans, sheep, horses, cattle, pigs, dogs, cats, rats, mice, birds, reptiles, fish, insects, and arachnids. The present definition of the term “bioactive” is specifically intended to exclude biocides utilized in inkjet applications.

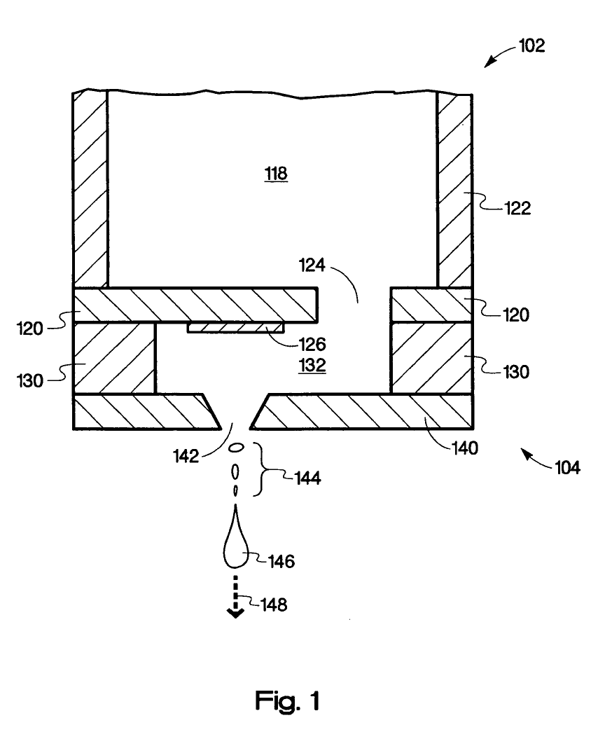

An embodiment of a fluid ejection system that may be utilized to prepare a bioactive dosage form according to the present invention is illustrated, in a cross-sectional view, in FIG. 1. In this embodiment, fluid reservoir 118 disposed in device body 122 of fluid ejection device 102, contains a first fluid that includes a reactant material. Fluid reservoir 118 is fluidically coupled to a substrate 120 via fluid inlet passage 124. Depending on the particular fluid ejection device utilized generally substrate 120 is attached to device body 122. However, in alternate embodiments, substrate 120 may include integrated circuitry and may be mounted to what is commonly referred to as a chip carrier (not shown), which is attached to device body 122. The substrate 120 generally contains an energy-generating element or fluid ejector 126 that generates the force utilized to eject essentially a drop of fluid held in chamber 132. Fluid or drop ejector 126 creates a discrete number of drops of a substantially fixed size or volume. Two widely used energy-generating elements are thermal resistors and piezoelectric elements. The former rapidly heats a component in the fluid above its boiling point causing vaporization of the fluid component resulting in ejection of a drop of the fluid, while the latter utilizes a voltage pulse to generate a compressive force on the fluid resulting in ejection of a drop of the fluid. For more information on various transducers utilized in drop-on-demand fluid ejection cartridges see, for example, Stephen F. Pond, Ph.D., Inkjet Technology and Product Development Strategies, ch 4 (Torrey Pines Research, 2000).

Substrate 120, chamber layer 130, nozzle layer 140, nozzles 142, form what is generally referred to as ejector head 104. Chamber layer 130 forms the side walls of chamber 132 and substrate 120 and nozzle layer 140 form the bottom and top of chamber 132 respectively, where the substrate is considered the bottom of the chamber. In this embodiment, fluid ejection device 102 has a nozzle density of 300 nozzles per inch; however, in alternate embodiments, nozzle densities may range from a single nozzle up to over a 1000 per inch. In addition, in this embodiment, nozzle layer 140 contains one nozzle per fluid ejector through which fluid is ejected; however, in alternate embodiments, each fluid ejector may utilize multiple nozzles through which fluid is ejected. Each activation of the fluid ejector results in the ejection of a precise quantity of fluid in the form of essentially a fluid drop with the drop ejected substantially along fluid ejection axis 148. Each fluid drop may include primary drop 146 as well as possible secondary drops 144. Both the generation and size of the secondary drops depends on various parameters such as the firing frequency of fluid ejector 126, the surface tension of the fluid being ejected, the size and shape of nozzle 142, and the size, shape, and location of fluid ejector 126 to nozzle 142. The number of times the fluid ejector is activated, in this embodiment, controls the number of drops ejected. In this embodiment, fluid ejection device 102 operates at a frequency of greater than 1 kilohertz for each fluid ejector or energy-generating element. Fluid ejection device 102 precisely controls in a discretely drop-by-drop manner the ejection of a fluid held in chamber 132. For more information on drop formation see, for example, Jaime H. Bohorquez et al., Laser-Comparable Inkjet Text Printing, Hewlett-Packard Journal, vol. 45, no. 1, pg. 9-17, Feb. 1994; or William A. Buskirk et al., Development of a High Resolution Thermal Inkjet Printhead, Hewlett-Packard Journal, vol. 39, no. 5, pg. 55-61, October 1988.

In this embodiment, the fluid ejection system also includes at least one additional fluid ejection device. The additional fluid ejection device includes a second fluid reservoir holding a second fluid that includes a co-reactant, which reacts with the reactant in the first fluid where either the first or second fluid also includes a bioactive fluid. However, in an alternate embodiment, the fluid ejection system may utilize a fluid ejection cartridge (not shown) having at least two fluid reservoirs, where each fluid reservoir is fluidically connected to one or more fluid ejector actuators that are fluidically isolated from the other fluid reservoirs.

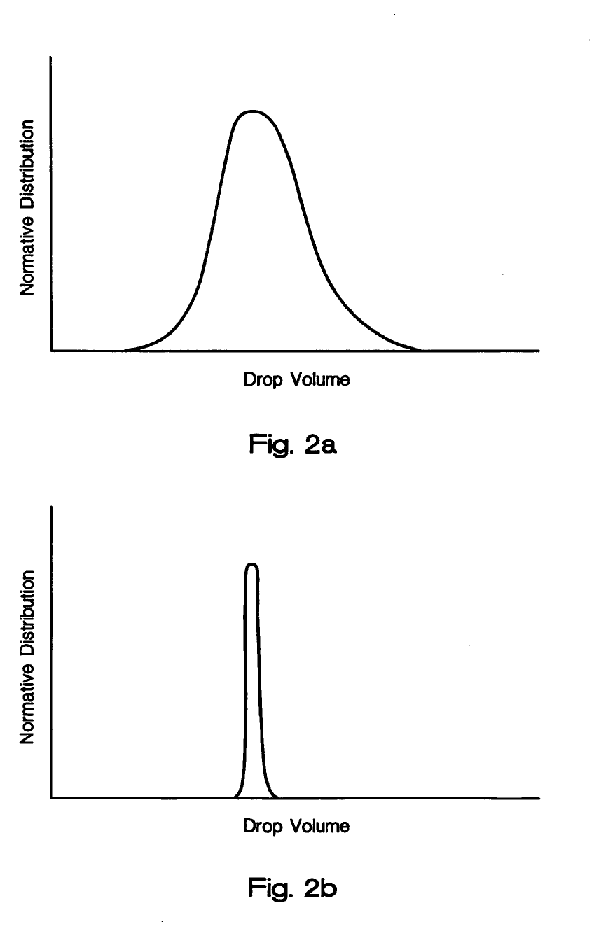

Fluid ejection device 102 described in the present invention can reproducibly and reliably eject drops in the range of from about 1 femto-liter to about ten pico-liters depending on the parameters of the fluid ejection device such as the size and geometry of the chamber around the fluid ejector, the size and geometry of the fluid ejector, and the size and geometry of the nozzle. In an alternate embodiment, utilizing what is generally referred to as a “direct drive” fluid ejection device, drops in the range from about 1 pico-liter to about 1 micro-liter also may be utilized. In addition, in still other embodiments, drops in the range from about 5 femto-liters to about 100 pico-liters also may be utilized. Fluid ejection device 102 differs from conventional fluid ejectors such as hydraulic, air assisted, or ultrasonic nozzles in that rather than forming a spray of fluid having varying drop sizes this embodiment utilizes a drop generator that creates fixed-sized drops that are discretely ejected. FIG. 2a is a graph describing the normalized distributed equivalent drop diameters for conventional fluid ejectors utilizing hydraulic, air assisted, or ultrasonic nozzles. The particular drop size distribution depends on the nozzle type and generally varies from one type to another. In addition, other factors such as the fluid properties, nozzle capacity, and spraying pressure also affect both the drop size and the drop distribution. As can be seen from FIG. 2a conventional fluid ejectors generally have a broad distribution of drop sizes. Fluid ejection device 102 differs from conventional fluid ejectors in that rather than forming a spray of fluid having varying drops sizes, activation of drop ejector 126 generates substantially fixed size drops that are discretely ejected. Fluid ejection device 102, on the other hand utilizes a method of creating discrete sized drops that are independently ejected from a particular nozzle utilizing a particular fluid ejector while maintaining a narrow drop size distribution as shown in FIG. 2b. In addition, the narrow drop size distribution is maintained over multiple nozzles each having a separate fluid ejector and fired independently or simultaneously. As can be seen comparing FIGS. 2a and 2b the present invention has a very narrow distribution of drop sizes and may have anywhere from a 2×, 3× or even more narrower drop size distribution than conventional fluid ejectors. In this embodiment, the range in drop volume is generally within 10 percent of the targeted or specified value and under steady state conditions is within about 6 percent of the targeted value. Because of the narrow (near uniform) distribution of ejected drops from fluid ejector device 102, the distribution of the size of the deposits formed on the ingestible sheet or substrate, formed from the ejected drops, have a corresponding narrow distribution in size. Thus, the present invention has the ability to accurately dispense a fluid including a reactant material component with a part per million to a part per billion accuracy. This is particularly advantageous when dispensing substances that have a high preparation cost. For example, materials such as certain proteins, peptides, hormones, antibiotics, and bioactive materials derived from some natural products in scarce supply may be effectively dispensed utilizing such a fluid ejection device. In addition, the accuracy and precision is advantageous when dispensing concentrated substances, such as pharmaceuticals with high potency.

Generally, for those applications involving oral administration of pharmaceuticals, the ingestible sheets are safely edible or ingestible, and do not have an objectionable “feel” in the mouth. In addition, the sheets, typically, dissolve or degrade in body fluids and/or enzymes. However, the sheets can be made of non-degradable materials that are readily eliminated by the body. Generally, the sheets are hydrophilic and readily disintegrate in water and typically the dissolution or disintegration of the sheets is enhanced at the pH of the fluids in the stomach or upper intestine. Further, ingestible sheets that minimize unintended interactions with the bioactive fluid dispensed on the sheets and sheets that minimize the release of any sheet component that would cause unintended interactions with the bioactive fluid upon dissolution of the sheet, are also desirable.

Additional properties of the ingestible sheet that are desirable are the ability to remain stable over extended periods of time, at elevated temperatures, and at high or low levels of relative humidity. In addition, it is also desirable that the ingestible sheets are generally a poor medium for the growth of microorganisms to reduce spoilage. Further, ingestible sheets that possess reasonable mechanical properties such as tensile strength and tear strength are desirable to allow the sheets to be processed through the various steps of fabrication of the final dosage form using methods such as are recognized in the art.

Ingestible sheets that can be utilized in the present invention can be one or a mixture of organic film formers generally classified into two broad categories, i.e. polymeric and paper. Examples of such film formers are starch (i.e. both natural and chemically modified) and glycerin based sheets with or without a releasable backing. Other examples include, proteins such as gelatin, cellulose derivatives such as hydroxypropylmethylcellulose and the like; other polysaccharides such as pectin, xanthan gum, guar gum, algin and the like; synthetic polymers such as polyvinyl alcohol, polyvinylpyrrolidone and the like. Examples of ingestible sheets or edible films that can be utilized are those that are based on milk proteins, rice paper, potato wafer sheets, and films made from restructured fruits and vegetables.

In particular, sheets or films made from restructured fruits and vegetables are advantageous were it is desirable to mask or modify the taste or smell of the bioactive fluid being delivered. Further, these restructured fruit and vegetable films also provide a convenient approach to encourage children to take various medications as well as providing a more pleasing and varied taste for various medications taken by adults. For more information on restructured fruit and vegetable films, see for example U.S. Pat. No. 5,543,164 and U.S. Pat. No. 6,027,758.

The form of the ingestible sheet or substrate that can be utilized in the present invention can be any of the forms generally recognized in the art such as those used for paper, cardboard, or polymeric films. The ingestible substrate such as a sheet or a roll, typically, is uniform in thickness and in width. Although the thickness will depend on the particular bioactive fluid being dispensed, the particular ingestible sheet being utilized, and the particular method of manufacture used; the thickness, typically, ranges from about 10 to about 350 microns.

The dosage forms produced in accordance with the present invention are eminently suited to span the range of production from individualized doses made in a home or hospital environment to the high speed high volume production in a pharmaceutical manufacturing environment. Thus, the particular width and length will not only depend on both the particular bioactive fluid being dispensed and the particular ingestible material being utilized, but more particularly on the particular method of manufacture utilized. Thus, the ingestible substrate can be in roll or individual sheet forms with widths varying from a few millimeters to several meters, and lengths from a few millimeters to several thousand meters, although other lengths and widths also may be utilized.

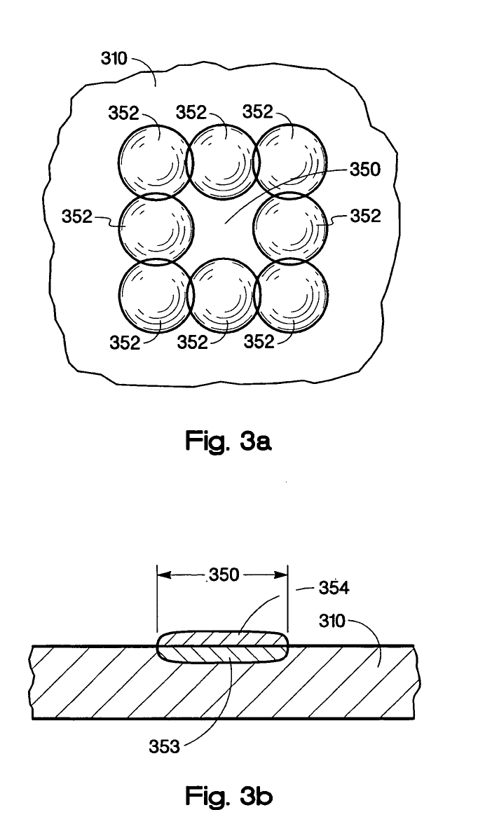

It has been found that by ejecting or jetting drops of two or more reacting fluids, each having a predetermined volume and at least one of which also includes a bioactive agent, onto a pre-selected location of the surface of an ingestible sheet precision dispensing of bioactive agents covering adjoining areas in a controllable manner can be obtained. In addition, utilization of such a system provides for such desirable effects as improved drytime, smearfastness, waterfastness, permanence, and cockle control. A wide variety of reactions may be utilized in the present invention, such as causing precipitation of a component, causing a change in pH, causing a change in viscosity, causing aggregation of a component, and forming a polymeric component. In one embodiment, shown in a plan view in FIG. 3a, fluid deposits 352 essentially border or surround receiving area 350. In this embodiment, fluid deposits 352 are formed on ingestible substrate 310 utilizing a first fluid that includes a reactant. A second fluid is subsequently deposited in receiving area 350, which includes a co-reactant to the reactant in the first fluid and a bioactive agent. The reaction between the reactant of the first fluid and the co-reactant of the second fluid substantially limits the lateral growth or spreading of the deposited drop of the bioactive agent in the second fluid. Such a system of fluid depositions provides a method for defining the area onto which a bioactive substance may be deposited.

In an alternate embodiment, shown in a cross-sectional view in FIG. 3b, first fluid deposit 353 is formed on ingestible substrate 310 from a first fluid that includes a reactant. Second fluid deposit 354 is formed over first fluid deposit 353. In this embodiment, second fluid deposit 354 includes a bioactive agent and a co-reactant to the reactant in first fluid deposit 353. For illustrative purposes only first fluid deposit 353 is shown as absorbing into ingestible substrate 310 indicative of an ingestible material having a porous or fibrous nature; however, this embodiment also may utilize non-absorbing non-porous or non-fibrous material as well. In such a case, first fluid deposit 353 forms on the surface of the ingestible material with little or no absorption into the media and second fluid deposit 354 forms on first fluid deposit 353. In this embodiment, the inclusion of the bioactive agent in the second fluid provides a method for controlling the lateral spread of the bioactive agent past the boundary formed by first fluid deposit 353. In an alternate embodiment, the bioactive agent may be included in first fluid deposit 353 in which case second fluid deposit 354 in reacting with the first fluid deposit provides for a protective layer over the bioactive agent. In still other embodiments, the first fluid may include the bioactive agent and first and second fluid deposits 353 and 354 are essentially simultaneously deposited on the ingestible substrate. In such a case, both lateral growth of the bioactive agent is substantially hindered and second fluid deposit also may form a protective layer over the bioactive agent. The dosage forms generated utilizing the present invention may include a wide variety of bioactive agents including virtually any drug, medication, medicament, vitamin, nutritional supplement, or other compound that is designed to affect a biological function. In addition, where mild conditions are desired to maintain the activity of biological molecules and macromolecules, the relatively mild conditions utilized in the present invention provide a method generating a dosage form including hemoglobin, cells, enzymes, or other biological molecules. Further, the present invention also may be utilized to generate dosage forms utilizing protein and peptide drugs that are susceptible to enzymatic attack and acidic hydrolysis in the gastrointestinal region if orally administered. Examples of proteins that may be utilized include interferons, interlukens darbepoetins, ethanercept, epogens, activases, and dornases. Examples of peptides that may be utilized include gonadotropins, lisinopril, calcitonin, ocreotide, leuprolide, and glucagons family peptides. Living cells such as streptococcus thermophilius, Bifidobactria, pancreatic cells, and red blood cells are just a few examples of living cells, with isotonic adjustment as needed, that may be utilized in the present invention.

A wide variety of reaction systems may be utilized to generate a pharmaceutical dosage form of the present invention. The particular process and reactants utilized will depend on various factors such as the particular bioactive agent used, and the particular ingestible sheet material used. In one embodiment, a complex coacervation process occurs where cationic and anionic water soluble polymers interact to form a polymer rich phase called a complex coacervate. Complex coacervation utilizes two oppositely charged polymers, i.e. a cationic and an anionic species where both species are incorporated into the dosage form. For example, a chitosan salt in a first fluid may be coupled with an aqueous based polymer in a second fluid to form a complex coacervate that can produce the desired effect of limiting the lateral spread of a bioactive agent when the first and second fluids are ejected onto an ingestible film material. Without being limited by theory, it is believed the chitosan salt and the polymer combine to form a gelatinous deposit where the two fluids overlap. The bioactive agent and water are entrapped in the gelatinous deposit. The bioactive agent may be added either to the first or second fluids depending on the particular bioactive agent and the particular ingestible sheet material utilized.

In another embodiment, the first fluid may include a monomer and a water insoluble bioactive agent dispersed using a dispersing agent. In this example, the second fluid includes a co-reactant to the monomer of the first fluid. The particular monomer utilized will depend on the particular bioactive agent used and the particular application in which the dosage form will be utilized. Various monomers such as isocyanates, esters, acids, aldehydes, ketones as well as combinations or mixtures of monomers all may be utilized. The particular co-reactant utilized depends on the particular monomer utilized in the first fluid. For example, a polyurea deposit is formed between an amine co-reactant and an acid monomer, whereas a polyamide is formed between an amine co-reactant and an acid ester monomer. A polyurethane deposit may be formed between the reaction of a hydroxyl containing co-reactant and an isocyanate monomer.

In an exemplary embodiment, chitosan (polyglucosamines, such as found in exoskeleton matter like crab shells) of approximately 5,000 MW (weight average) in solution can be combined with certain polymers to form a gel deposit on an ingestible substrate, such as rice paper, potato wafer sheets, and starch based sheets. Examples of suitable salts of chitosan include chitosan acetate, chitosan lactate, and chitosan succinate. By “chitosan” or “chitosan salts” as used herein, is also meant the broader class of reactive polymers based on chitosan, polysaccharides, and oxidized glucose, including polyglucosamines, polysaccharides modified with cationic functionalities, and polysaccharides modified with carboxylate or other anionic functionalities, e.g. carboxy methyl chitosan. Other suitable charged polysaccharides included under the general term “chitosan,” as used herein, include chonfrotin sulfate, available from Vanson, Inc., Morristown, N.J. as Polychon™, carboxymethyl cellulose, hyaluronic acid-N-acetyl d-glucosamine and D-glucoronic acid polymer, alginates, alginic acid-1,4 linker polymer of D-mannonuronic acid (D-mannose is a saccaride), carrageenans (with sulfate content of approximately 15%), and dextran sulfate. Suitable cationic polymers include diethyl amonoethyl cellulose (available as celquat H-100, L-200™ from National Starch Co.), dextran (DEZE™), cationic guars available from Celenese as Jaguars C-14s™, C-15s™ and C-17™, Cationic starch, such as cato-72™, from National Starch, and cellulose/starch-dimethylallyl ammonium chloride copolymers, such as Floc-Aid 19™ from National Starch.

The chitosan salt, which may be utilized either alone or in any combination, is present in one of the fluids in the range from about 0.1 weight percent to about 20 weight percent. In an alternate embodiment, less than about 0.5 weight percent is utilized. The reactive fluid, in addition to water and the chitosan salt described above, may also contain one or more solvents, surfactants, amphiphiles, or buffers. The second reactive fluid includes a set of one or more polymers matched to the chitosan salt utilized in the first reactive fluid. Examples of such polymers include polyacrylic acid (MW of approximately 5,000), polystyrenemaleic anhydride derivatives, rosin, polyabiatic acid-maleic anhydride derivatives, polyamides such as GAX-12-513 from Henkel, polyolefin-acrylates, styrenated polyacrylates such as GAX-600 from Henkel, and ABC triblock polymers, wherein A block is a water soluble hydrophilic polymer, B block has functional groups, and C block is a polymer which is soluble in at least one water soluble organic solvent. Examples of ABC triblock polymers include methacrylic acid//phenylethylmethacrylate/dimethylaminoethyl methacrylate//ethoxytriethylene glycol methacrylate (13//8/2//4), dimethylaminoethyl methacrylate/methyl methacrylate//phenylethyl methacrylate//ethoxytriethylene glycol methacrylate (7.5/5//10//4), and methacrylic acid//phenylethyl methacrylate//ethoxy-triethylene glycol methacrylate (13//10/4). The polymer which may be utilized either alone or in any combination, is present in the second reactive fluid in the range from about 0.1 weight percent to about 10 weight percent. The second reactive fluid, in addition to water and the polymer described above, also may contain one or more solvents, surfactants, amphiphiles, or buffers. These other ingredients that may be added to the two reactive fluids of the present invention should be compatible with the bioactive agent utilized as well as with the above reactive agents utilized.

A wide variety of bioactive substances may be utilized to generate a pharmaceutical dosage form of the present invention. Any bioactive substance that is soluble or dispersible in a suitable fluid for use in a fluid ejection device may be utilized. Examples of bioactive substances that may be utilized in the present invention include ace inhibitors such as enalaprilat and trandolapril; alpha agonists/alpha blockers such as reserpine and yohimbine hyrodchloride; general analgesics such as buprenorphine hydrochloride and sarracenia purpurea; antianxiety such as clidinium bromide; antiarthritics such as auranofin; antiasthmatics/broncodilators such as carbinoxamine maleate and loratadine; antidiarrheals such as difenoxin hydrochloride; antidiuretics such as desmopressin acetate; specific antidotes such as phenyl salicylate; antihistamines such as desloratadine, phenindamine tartrate, tripelennamine hydrochloride, and triprolidine hydrochloride; antihypertensives such as bendroflumethiazide, candesartan cilexetil, deserpidine, diazoxide; trichlormethiazide; antimigraine such as ergotamine tartrate, and tegaserod maleate; antineoplastics such as idarubicin hydrochloride, melphalan, and tamoxifen; antipsychotics/antimanics such as risperidone; antitussives/expectorants/mucolytics such as carbetapentane; calcium channel blockers such as nisoldipine; acid/peptic disorders such as cisapride and famotidine; extrapyramidal movement disorders such as bromocriptine; hemostatics such as phytonadione; hyperlipidemia such as ezetimibe and lovastatin; immunomodulators such as tacrolimus; metabolics/nutrients such as rosuvastatin calcium; myasthenia gravis such as ambenonium chloride; relaxants/stimulants, uterine such as ergonovine maleate; and milrinone lactate.

The fluids of the present invention, both the first and second reactant fluids, may comprise from about 0.1 to about 40 weight percent of at least one organic solvent. Optionally, one or more water-soluble surfactants, amphiphiles or combinations thereof may be present from 0 to about 10 weight percent. Other ingredients added to the reactant fluids of this invention should be compatible with the particular bioactive substance or substances employed in this invention as well as the particular reactive agent employed.

The aqueous vehicle is water or a mixture of water and at least one water-soluble organic solvent. Selection of a suitable mixture depends on the requirements of the specific application, such as the desired surface tension and viscosity, the selected bioactive material, the selected reactive agent, and the type of medium or ingestible substrate onto which the fluids are ejected.

Water soluble organic solvents that may be suitably employed in the present invention include any of, or a mixture of two or more, of such compounds as nitrogen containing ketones, such as 2-pyrrolidinone, N-methyl-2-pyrrolidinone (NMP), 1,3-dimethylimidiazol-2-one, and octyl-pyrrolidinone; diols such as ethanediols (e.g. 1,2-ethanediol), propanediols (e.g. 1,2-propanediol, 1,3-propanediol), butanediols (e.g. 1,2-butanediol, 1,3-butanediol, 1,4 butanediol), pentanediols (e.g. 1,2-pentanediol, 1,5-pentanediol), hexanediols (e.g. 1,2-hexanediol, 1,6-hexanediol, 2,5-hexanediol), heptanediols (e.g. 1,2-heptanediol, 1,7-heptanediol), octanediols (e.g. 1,2-octanediol, 1,8-octanediol); triolos such as 2-ethyl-2-hydroxymethyl-1,3-propanediol and ethylhydroxypropanediol (EHPD); and glycol ethers and thioglycol ethers such as polyalkylene glycols such as polyethylene glycols (e.g. diethylene glycol (DEG), triethylene glycol, tetraethylene glycol), polypropylene glycols (e.g. dipropylene glycol, tripropylene glycol, tetrapropylene glycol, polymeric glycols (e.g. PEG 200, PEG, 300, PEG 400, PPG 400) and thiodiglycol.

Suitable surfactants may be nonionic or anionic when used in the fluid vehicle. Examples of suitable nonionic surfactants include, secondary alcohol ethoxylates (e.g. Tergitol series available from Union Carbide Co.), nonionic fluoro surfactants such as FC-170C available from 3M, nonionic fatty acid ethoxylate surfactants (e.g. Alkamul PSMO-20 available from Rhone-Poulenc), fatty amide ethoxylate surfactants (e.g. Aldamide L-203 available from Rhone-Poulenc), and acetylenic polyethylene oxide surfactants (e.g. Surfynol series, available from Air Products & Chemicals, Inc.). Examples of anionic surfactants include alkyldiphenyloxide surfactants (such as Calfax available from Pilot), and Dowfax (e.g. Dowfax 8390 available from Dow Chemical), and fluorinated surfactants (Fluorad series available from 3M). Cationic surfactants that may be utilized include betaines (e.g. Hartofol CB-45 available from Hart Product Corp., Mackam OCT-50 available from McIntyre Group Ltd., Amisoft series available from Ajinomoto), quartenary ammonium compounds (e.g. Glucquat series available from Amerchol, Bardac and Barquat series available from Lonza), cationic amine oxides (e.g. Rhodamox series available from Rhone-Poulenc), Barlox series available from Lonza), and imidazoline surfactants (e.g. Miramine series available from Rhone-Poulenc, Unamine series available from Lonza).

Buffers can be used to modulate the pH of the fluids. They may be organic based biological buffers or inorganic buffers such as sodium phosphate. Furthermore, the buffer employed should provide a pH ranging from about 3 to about 9 in the practice of the invention. Examples of organic buffers that may be utilized in the present invention include Trizma base, available from companies such as Aldrich Chemical (Milwaukee Wis.), 4-morpholinoethanesulfonic acid (MES) and 4-morpholinopropanesulfonic acid (MOPS).

The balance of the fluid compositions of the present invention comprises water, specifically, deionized water. The first and second reactant fluids within the foregoing listed ranges may be ejected on a wide variety of ingestible substrates as discussed above. In addition, additional energy may be provided to the reaction to increase the benefits of the chitosan-polymer reaction. For example, thermal energy may be added by heating the substrate utilizing a drum or fuser. Photolytic energy also may be utilized by using a light bar or laser of the appropriate wavelength. Chemical treatment with a suitable organic or inorganic acid or base of the dosage form also may be utilized.

What is claimed is:

1. A system for generating a bioactive dosage form, comprising:
a first drop-on-demand fluid ejector fluidically coupled to a first reservoir, said first reservoir containing a first fluid having a first reactant, said first drop-on-demand fluid ejector adapted to eject a drop of said first fluid onto a pre-selected location of an ingestible substrate; and
a second drop-on-demand fluid ejector fluidically coupled to a second reservoir, said second reservoir containing a second fluid having a co-reactant which reacts with said first reactant, wherein either said first fluid or said second fluid contains a bioactive agent.

2. The system in accordance with claim 1, wherein said first reactant is a chitosan salt.

3. The system in accordance with claim 2, wherein said chitosan salt is selected from the group consisting of chitosan acetate, chitosan lactate, chitosan succinate, polyglucoamines, polysaccharides modified with cationic or anionic functionalities, and mixtures thereof.

4. The system in accordance with claim 1, wherein said second reactant further comprises a polymeric agent selected to react with chitosan to form a gel precipitate.

5. The system in accordance with claim 1, wherein said second reactant further comprises a polymeric agent.

6. The system in accordance with claim 5, wherein said polymeric agent is selected from the group consisting of polyacrylic acid, polystyrene-maleic anhydride derivatives, rosin, polyabiatic acid-maleic anhydride derivatives, polyamides, polyolefin-acrylates, styrenated polyacrylates, ABC triblock polymers, and mixtures thereof.

7. The system in accordance with claim 1, wherein said first reactant further comprises a polyanion.

8. The system in accordance with claim 1, further comprising a reaction enhancing device configured to provide additional energy to said reaction.

9. The system in accordance with claim 1, wherein said bioactive agent is selected from the group consisting of hemoglobin, a red blood cell, a living cell, a protein, a peptide, and mixtures thereof.

10. The system in accordance with claim 1, wherein said bioactive agent is selected from the group consisting of alpha agonists, alpha blockers, analgesics, anti-arthritics, anti-asthmatics, anti-diarrheals, anti-diuretics, antihistamines, anti-hypertensives, anti-migraines, anti-neoplastics, anti-psychotics, anti-tussives, and mixtures thereof.

11. The system in accordance with claim 1, wherein said first reactant and said second reactant react upon contact on said ingestible substrate.

12. The system in accordance with claim 1, wherein said ingestible substrate is formed from a restructured fruit or vegetable.

13. The system in accordance with claim 1, wherein said ingestible substrate includes an organic film former.

14. The system in accordance with claim 1, wherein said first drop-on-demand fluid ejector produces a distribution of drop volumes within 10 percent of a specified volume.

15. A method of making a pharmaceutical dosage form, comprising:
activating a first drop-on-demand fluid ejector to eject essentially a drop of a first fluid including a first reactant onto an ingestible substrate;
activating a second drop-on-demand fluid ejector to eject essentially a drop of a second fluid including a bioactive agent and a co-reactant of said first reactant on said ingestible sheet at least proximate to said drop of said first fluid; and
reacting said first reactant with said co-reactant.

16. The method of making a pharmaceutical dosage form in accordance with claim 15, wherein said first reactant is a chitosan salt.

17. The method of making a pharmaceutical dosage form in accordance with claim 16, wherein said chitosan salt is selected from the group consisting of chitosan acetate, chitosan lactate, chitosan succinate, polyglucoamines, polysaccharides modified with cationic or anionic functionalities, and mixtures thereof.

18. The method of making a pharmaceutical dosage form in accordance with claim 16, wherein said second reactant further comprises a polymeric agent selected to react with said chitosan salt forming a gel precipitate.

19. The method of making a pharmaceutical dosage form in accordance with claim 15, wherein said second reactant further comprises a polymeric agent.

20. The method of making a pharmaceutical dosage form in accordance with claim 19, wherein said polymeric agent is selected from the group consisting of polyacrylic acid, polystyrene-maleic anhydride derivatives, rosin, polyabiatic acid-maleic anhydride derivatives, polyamides, polyolefin-acrylates, styrenated polyacrylates, ABC triblock polymers, and mixtures thereof.

21. The method of making a pharmaceutical dosage form in accordance with claim 15, wherein said first reactant further comprises a polyanion.

22. The method of making a pharmaceutical dosage form in accordance with claim 15, further comprising subjecting said first reactant and said second reactant to additional energy where said first and second reactants are in contact.

23. The method of making a pharmaceutical dosage form in accordance with claim 22, wherein said additional energy is selected from the group consisting of thermal energy, photolytic energy, chemical energy, and combinations thereof.

24. The method of making a pharmaceutical dosage form in accordance with claim 15, wherein activating a first drop-on-demand fluid ejector further comprises depositing said first fluid onto said ingestible substrate in a pre-selected pattern substantially enclosing a receiving area.

25. The method of making a pharmaceutical dosage form in accordance with claim 24, wherein activating said second drop-on-demand fluid ejector further comprises depositing said second fluid substantially in said receiving area.

26. The method of making a pharmaceutical dosage form in accordance with claim 15, wherein activating said second drop-on-demand fluid ejector further comprises depositing said second fluid substantially over said drop of said first fluid.

27. The method of making a pharmaceutical dosage form in accordance with claim 15, wherein activating said first drop-on-demand fluid ejector further comprises activating said first drop-on-demand fluid ejector n times, ejecting n drops of said first fluid onto said ingestible substrate, wherein n is an integer.

28. The method of making a pharmaceutical dosage form in accordance with claim 27, wherein said n drops produce a distribution of drop volumes within 10 percent of a specified volume.

29. The method of making a pharmaceutical dosage form in accordance with claim 27, further comprising activating said first drop-on-demand fluid ejector at a steady state producing a distribution of drop volumes within 6 percent of a specified volume.

30. A method of making a pharmaceutical dosage form, comprising:
printing a first fluid comprising a first reactant onto an ingestible fluid receiving medium;
printing a second fluid comprising a second reactant which reacts with said first reactant where said second reactant contacts said first reactant, wherein at least one of said fluids further comprises a bioactive agent.

31. A method of using a drop on demand fluid ejection device, comprising:
energizing the drop-on-demand fluid ejection device;
ejecting essentially a first fluid drop including a first reactant component onto an ingestible sheet; and
ejecting essentially a second fluid drop including a second reactant component onto said ingestible sheet wherein either said first fluid or said second fluid further comprises a bioactive agent.

32. A system for generating a pharmaceutical dosage form, comprising:
an ingestible substrate;
a fluid set having at least two or more fluids wherein
a) at least one of said fluids comprises a bioactive agent;
b) at least one of said fluids comprises a first reactant; and
c) at least one of said fluids comprises a second reactant which reacts with said first reactant. whereby the pharmaceutical dosage form is generated by dispensing said at least two or more fluids on said ingestible sheet and reacting said first and second reactants.
